SEQUENCE LISTING (15 sequences)

SEQ ID NO: 1 - DNA, 1551 bp, Homo sapiens
  sig_peptide (1)..(57)
atggctggcagcctcacaggtttgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcatcgccagtga

SEQ ID NO: 2 - protein, 516 aa, Homo sapiens
MAGSLTGLLLLQAVSWASGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQ

SEQ ID NO: 3 - DNA, 27 bp, Artificial Sequence
  forward primer for human GCB amplification including DNA coding for native signal peptide
tctagaatggctggcagcctcacaggt

SEQ ID NO: 4 - DNA, 33 bp, Artificial Sequence
  Reverse primer for human GCB amplification
gtgtggatggacaccgtagcggtcactctcgag

SEQ ID NO: 5 - DNA, 31 bp, Artificial Sequence
  forward primer for human GCB amplification excluding DNA coding for native signal peptide
cccgggtgcccgcccctgcatccctaaaagc

SEQ ID NO: 6 - DNA, 1428 bp, Glycine max
  promoter (1)..(1428)
taaaataatctatacattaaaaaatttgattttaaaattttagaaattcatgattttatttttttttaccagaaatccgttaatattgttaaaatattaccaactaatttataaattttattttaaggcaattaagcatgtttgataaaatatatatattgttataaatacttttcaaaagtataaagttgatgatggcgtggtggtagattattttagttctaggttcgaatgcaagttggtttagacatttagccttattcttttttctaaccaaaataaatgtaaatggaaaacctttaggaaaaaaaagaaatcaaaattgaaaacatcatccggtggagtcgagaagcccacacccacgtgacccaacaatattaaaataagagtttgctctacagtaaatgcgatacttttttattcaatactttttccacttctaaaatcttggagatttgcaccgttaactaattaagtgttatatccaacggtcctaaaaaaacttgtgtaccgtgcctcacatttcaactttgcgcaccctagaagccgtctatgtttaggttagtgtttgcaacagttgaagcgcatcactcaggaggctacttggtcttgcttttgcgtcttttgttcaatttttcacgtgattttgttggtgaacacgcgtacttgaaacttattataaattacataattttataagtttcacttcttatataatacttcattcatgcatttataattttgatgaataataaagagtttgttaaaaaatatattatttcatataatatatagggtttagaatgccaatttttaaaaaaagaataaaaaaataaatagaataaaatcgaaaaaatgaaatgtaaaaaatttgagggggacaaataaaatatgaaagtctattatttaaattttccattagaattctattttccttagttaatatgagctagccagttgggagatacacgaaaatgtcatgaaacagttgcatgtagggaaattaatgtagtagagggatagcaagacaaaaatccaagccaagctagctgctcacgcgaactcgatccacacgtcctttacagagtttcaaacggatgaaatctgcatggcatgcaactaaagcattgttctcagctgccaagtacccctcacactcaccaaccctttgtttttctccccattgcatgttaactcaagtttatcctttctttgcttctggaaatttcacaagcctcaaacacgtcgacgtccaatcttgtgaccaacacggccaaaagaaaagagaatctcatcccgttcacacttagccacttaaagctagccaaacggtgatctttctctatatattgtagctctctaacacaaccaacactaccattattcaatattcaaaccttgctctatactacacacactagaagaata

SEQ ID NO: 7 - DNA, 72 bp, Glycine max
  sig_peptide (1)..(72)
atggcttctatcctccactactttttagccctctctctttcttgctcttttcttttcttcttatccgactca

SEQ ID NO: 8 - DNA, 1319 bp, Homo sapiens
  sig_peptide (21)..(116)
aatgctgtccggtcaccgtgacaatgcagctgaggaacccagaactacatctgggctgcgcgcttgcgcttcgcttcctggccctcgtttcctgggacatccctggggctagagcactggacaatggattggcaaggacgcctaccatgggctggctgcactgggagcgcttcatgtgcaaccttgactgccaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagtagatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttaaaatgtt

SEQ ID NO: 9 - protein, 430 aa, Homo sapiens
TMQLRNPELHLGCALALRFLALVSWDIPGARALDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLL

SEQ ID NO: 10 - DNA, 29 bp, Artificial Sequence
  forward primer for human GLA amplification excluding DNA coding for native signal peptide
ggatccctggacaatggattggcaaggac

SEQ ID NO: 11 - DNA, 33 bp, Artificial Sequence
  reverse primer for human GLA amplification
gtctacagtaattttctgaatgaaattctatag

SEQ ID NO: 12 - DNA, 3624 bp, Homo sapiens
  sig_peptide (220)..(426)
cagttgggaaagctgaggttgtcgccggggccgcgggtggaggtcggggatgaggcagcaggtaggacagtgacctcggtgacgcgaaggaccccggccacctctaggttctcctcgtccgcccgttgttcagcgagggaggctctgggcctgccgcagctgacggggaaactgaggcacggagcgggcctgtaggagctgtccaggccatctccaaccatgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgctgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcgtgtccacagccgggcaccgtccccactctacagcgtggagttctccgaggagcccttcggggtgatcgtgcaccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccaatggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcagaggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccaatagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggagaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttagccgggcggagtgtgttagtctctccagagggaggctggttccccagggaagcagagcctgtgtgcgggcagcagctgtgtgcgggcctgggggttgcatgtgtcacctggagctgggcactaaccattccaagccgccgcatcgcttgtttccacctcctgggccggggctctggcccccaacgtgtctaggagagctttctccctagatcgcactgtgggccggggcctggagggctgctctgtgttaataagattgtaaggtttgccctcctcacctgttgccggcatgcgggtagtattagccacccccctccatctgttcccagcaccggagaagggggtgctcaggtggaggtgtggggtatgcacctgagctcctgcttcgcgcctgctgctctgccccaacgcgaccgcttcccggctgcccagagggctggatgcctgccggtccccgagcaagcctgggaactcaggaaaattcacaggacttgggagattctaaatcttaagtgcaattattttaataaaaggggcatttggaatc

SEQ ID NO: 13 - protein, 883 aa, Homo sapiens
AHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPRVHSRAPSPLYSVEFSEEPFGVIVHRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPIEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 14 - DNA, 27 bp, Artificial Sequence
  forward primer for human GAA amplification excluding DNA coding for native signal peptide
gatatctgcacaccccggccgtcccag

SEQ ID NO: 15 - DNA, 30 bp, Artificial Sequence
  reverse primer for human GAA amplification
gtcaaagagcagtcgaccacaatcctatag

(19) United States
(12) Patent Application Publication
(10) Pub. No.: US 2006/0031965 A1
(43) Pub. Date: Feb. 9, 2006

(54) In- seed expression of lysosomal enzymes

(76) Inventors: Corrado Fogher, Casalmaggiore (IT); Serena Reggi, Piacenza Pc (IT)

Correspondence Address: BEUSSE BROWNLEE WOLTER MORA & MAIRE, P. A., 390 NORTH ORANGE AVENUE, SUITE 2500, ORLANDO, FL, 32801, US

(21) Appl. No.: 10/506,448

(22) PCT Filed: Mar. 3, 2003
(86) PCT No.: PCT/IT03/00120

§ 371 Date: Sep. 1, 2004

(30) Foreign Application Priority Data
Mar. 1, 2002 (IT) RM2002A000115

(51) Int. Cl. A01H 1/00; C12N 15/82; C12N 5/04

(52) U.S. Cl. 800/288; 435/419; 435/468

(57) ABSTRACT

The present invention relates to the production of transgenic plants able to express, in seed storage tissues, a lysosomal enzyme in enzymatically active form and in amounts appropriate to its use in enzyme replacement therapy. In particular, the present invention relates to plants and seeds containing this enzyme.

### FIELD OF THE INVENTION

The present invention relates to the production of transgenic plants able to express, in seed storage tissues, a lysosomal enzyme in enzymatically active form and in amounts appropriate to its use in enzyme replacement therapy.

In particular, the present invention relates to plants and seeds containing this enzyme.

### PRIOR STATE OF THE ART

Lysosomal diseases represent a wide class of human genetic diseases determined by malfunctions of specific lysosomal enzymes. Lysosomes are the main degradative organelles of animal cells and are of critical importance in degrading macromolecules and in recycling their monomeric components. These membrane-delimited organelles exhibit acidic pH and contain a variety of nucleases, proteases, phosphatases and degradative enzymes for polysaccharides, mucopolysaccharides and lipids. A malfunction of a specific acid hydrolase determines an aberrant accumulation of the substrate in the lysosomes, causing a variety of pathologies, among which there may be mentioned: Tay-Sachs disease, due to a deficiency of the enzyme β-N-hexosaminidase, Mucopolysaccharidoses (MPSs) a group of recessive disorders due to a malfunction in the degradation of complex sulphurates, Anderson-Fabry disease, due to a deficiency of the enzyme α-galactosidase A causing accumulation of globotriaosylceramide mostly in renal microvascular endothelial cells, Pompe disease, due to a deficiency of the enzyme acid α-glucosidase leading to intralysosomal accumulation of glycogen and Gaucher disease, due to deficiences in the enzyme glucocerebrosidase (acid β-glucosidase or GCB) that determines the accumulation of glycosphingolipids mainly in cells of monocyte-macrophage lines.

In particular, Anderson-Fabry disease is a dominant X-linked disease resulting from the malfunction of the enzyme α-galactosidase A (α-GalA or GLA), which leads to the progressive accumulation of globotriaosylceramide (GL-3) and of the related glycosphingolipids causing proteinuria in young males and subsequently, with age, kidney failure.

Human α-galactosidase A is a 429 amino acids (aa) homodimeric glycoprotein, with four putative glycosylation sites, whose signal peptide is represented by the 31 N-terminal residues. The cDNA sequence of GLA has been published by Tsuji S. et al., 1987, Eur. J. Biochem., 165(2), 275-280.

Pompe disease, or glycogen storage disease type II, is an autosomal recessive metabolic myopathy caused by a deficiency of the enzyme acid β-glucosidase (GAA) which leads to storage of glycogen in almost all tissues, specifically injuring heart and skeletal muscles.

Acid β-glucosidase is a 952 aa glycoprotein having seven putative glycosylation sites, whose signal peptide is represented by the 69 N-terminal residues. cDNA sequence has been published by Hoefsloot L. H. et al., 1988, EMBO JOURNAL 7(6), 1697-1704.

Lastly, Gaucher disease is an autosomal recessive disorder caused by a deficiency of glucocerebrosidase, an enzyme required for degradation at lysosomal level of lipids containing covalently bonded sugars (Brady et al. 1965, J. Biol. Chem., 240: 39-43). In the absence of glucocerebrosidase, the insoluble compound glucocerebroside (glucosylceramide) accumulates in the lysosomes leading to the disease symptomatology.

Human glucocerebrosidase is a glycoprotein having a molecular weight ranging from 58 to 70 kDa, with five putative glycosylation sites. The complete cDNA has been reported (Sorge et al., 1985, Proc. Natl. Acad. Sci. 82: 7289-7293). The open reading frame, having 1545 base pairs (bp) plus several introns, codes for a protein having 515-amino acid, the 19 amino acids of the peptide signal included.

Success attained by enzyme replacement therapy in lysosomal diseases like Gaucher disease underlined the demand for more effective, safe and economical production methods of lysosomal enzymes in general.

To date, some of these enzymes have been directly extracted from human placenta or produced, by genetic engineering, in CHO (Chinese hamster ovary) cell cultures. Both of the abovementioned production methods, besides being very costly, entail risks for the patient. In fact, following the use of placenta-extracted enzymes, entailed viral infections have been observed, whereas following the use of enzymes extracted from CHO cell cultures, in the 15% of the patients production of specific antibodies against the product was detected, whose origin could relate to the production system adopted.

Only recently some lysosomal enzymes have been produced in genetically modified plants.

Human lysosomal enzymes can be produced in transgenic plants in order to solve problems of safety, viral infections, immune reactions, production yield and cost.

U.S. Pat. No. 5,929,304 reports the “in-leaf” production of some lysosomal enzymes (human glucocerebrosidase and human α-L-iduronidase), optimally generated in tobacco.

In fact, tobacco is particularly indicated as a model system for the production of high-value recombinant proteins. Heterologous genes can be introduced in totipotent cells of tobacco using nonpathogenic strains of Agrobacterium tumefaciens. The subsequent growth and differentiation of the transformed cells leads to the attainment of stable transgenic plants. Large amounts of leaf material from transgenic plants are yielded in 6-7 weeks, whereas first-generation transgenic seeds, in amounts vastly greater than those of the leaf material, become available in other 2-3 months.

U.S. Pat. No. 5,929,304 describes the expression of some lysosomal enzymes in tobacco plants. The enzymes are produced essentially in leaf by plants transformed via the use of vectors containing the MeGa promoter (deriving from the tomato HMG2 promoter) or the cauliflower mosaic virus (CaMV) 35S promoter. U.S. Pat. No. 5,929,304 reports, among the promoters indicated as useful in carrying out the invention, besides the abovecited ones, also the rbcS promoter, the chlorophyll-binding a/b protein, the AdhI promoter and the NOS promoter. According to the teachings of this latter patent, the promoter used may be constitutive or inducible. In-leaf expression of enzymatically active human □-L-iduronidase is described in all the examples reported in the patent. The expression occurs following the transformation of tobacco plants with vectors containing, besides the sequence coding for the desired enzyme, the mechanically inducible MeGA promoter, or the constitutive 35S promoter, respectively.

As reported in the examples, the yield of human glucocerebrosidase in tobacco leaves is equal to about 1.5 mg of extractable protein per 1000 mg of tissue, i.e. the yielded enzyme is equal to the 0.15% b/w of the initial leaf material. This result, obtained by in-plant expression, has been indicated by the inventors as the long-sought solution for the production of animal or human lysosomal enzymes, thereby meeting the demand for said enzymes in replacement therapy.

However, in the leaf protein concentration is known to be extremely low, and anyhow lower than the one in seed. Moreover, it has to be borne in mind that in the case of in leaf enzyme expression there subsists a stability problem, due to the fact that enzymes are proteins extremely sensible to denaturating factors. Said stability problem requires the continual and prompt harvesting of the leaf tissue after the mechanical induction of the promoter, the immediate freezing at −20° C. of said tissue and the discarding of an enormous amount of material in the enzyme-extracting step. Moreover, the in-leaf expression allows production of a very low amount of proteins, anyhow lower with respect to an in seed production of the same. Hence, it would be useful to provide a seed-specific system for the expression of lysosomal enzymes.

Although the teachings of U.S. Pat. No. 5,929,304 theoretically relate also to the expression of the enzyme in plant tissues other than the leaf one, it has been verified by the present Inventors, while attempting at expressing acid β-glucosidase in seed, that the 35S promoter identified in said patent as one of the promoters suitable for this purpose is unable to direct in-seed accumulation of a stable protein (FIG. 12). As it is subsequently reported in the comparative example, tobacco plants were transformed using an expression vector comprising the cDNA sequence of the human glucocerebrosidase gene, coding also for the native signal sequence of the enzyme, under the control of the 35S promoter. Western Blot analysis under chemiluminescence demonstrated the absence of the desired enzyme from seed tissues. Hence, U.S. Pat. No. 5,929,304, although declaring the described method as suitable for in-seed expression, does not provide the teachings required in order to carry out said expression to a person skilled in the art.

In Seed Expression of Heterologous Proteins

Overall, seed constitutes the vegetal organ most widely used by humans for its caloric and protein contributions. Storage function for the nitrogen component is carried out by specific proteins accumulated in protein bodies, inside compartments in endocellular membrane systems. In seed, protein amounts range from about the 10-15% b/w in cereals to about the 25-35% in Leguminosae, whereas in tobacco they are equal to about the 20% b/w. Hence, protein accumulation should be directed in seed endosperm in order to increase the production yields of in plant expressed proteins.

International Patent Application WO-A-00/04146, to the same applicant, described the in-seed expression of protein lactoferrin. This result was obtained by modifying tobacco and rice plants with vectors containing an expression cassette. The cassette comprises a promoter, and a DNA sequence coding for the signal sequence of storage proteins highly abundant in seeds and stage-specifically expressed, i.e. βconglycinine or basic S7 globulin, and a DNA sequence coding for lactoferrin. WO-A-00/04146 details the vectors used and the promoters selected.

Lactoferrin is a glycoprotein able to bind iron and usually expressed in human milk. This protein exhibits an extremely high stability, given that lactoferrin-containing solutions may be treated even for 5 min at 90° C. without detecting significant losses in protein activity. Pat. Appln. WO00/04146 advances, by way of hypothesis, also the expression of proteins exhibiting a generically enzymatic activity using the method practiced with lactoferrin. However, this possibility remains purely theoretical, as no data is provided supporting the validity of the system adopted in said Patent Application for the expression of functionally active enzymes, even less so of lysosomal enzymes.

Pat. Appln. WO-A-00/04146, it being actually not aimed to enzyme expression, neglects several problems of fundamental relevance related to the stability of the hexogenous protein expressed. In fact, the utmost stability of lactoferrin and the fact that in lactoferrin the presence of non-natural glycosidic chains does not influence protein folding and function, definitely bars the use of the system disclosed in Pat. Appln. WO-A-00/04146 from effectively expressing enzymes exhibiting a correct folding and being functionally equivalent to the native enzyme. Moreover, it is known that in the case of the acid □-glucosidase the glycosylation of the first of the five glycosylation sites in the enzyme is crucial to the generation of an active enzyme; said function pertains to the third glycosylation site in the case of □ galactosidase A, and in the case of acid β-glucosidase, the second glycosylation site is the one crucial to the generation of the mature enzyme.

Another problem lies in that the protein instability could be due to structural restraints, but also be a consequence of its subcellular location. For the expression of lysosomal enzymes the fact should be taken into account that plant cells do not possess lysosomes, using the vacuole as a functional analogue of these acid vesicles for polymer degradation. In humans, the main pathway for transferring soluble enzymes to lysosomes implies the interaction with mannose-6-phosphate receptors (MPR). Lysosomal proteins are dispatched to the endoplasmic reticulum (RE) by N-terminal signal peptides, and are glycosilated during their transfer to Golgi apparatus. In this organelle, the N-linked glycans of the enzymes destinated to the lysosomes are selectively phosphorylated to one or more mannose residues. Then, the mannose-6-phosphate can bind to the membrane receptors which ensure glycoprotein internalization.

Plants lack the targeting system based on mannose-6-phosphate, do not possess MPRs and do not apppear to produce phosphorylated glycans. Since lysosomal enzymes function in an acidic environment (pH <4) and are unstable at higher (≧7) pH values, the pH (5.8) of the plant (extracellular) apoplastic compartment makes the secretion of said enzymes in said compartment ideal for their stability. Although it is inferrable that human lysosomal glycoproteins may be secreted in the apoplastic space (which would be particularly suitable for the storage of lysosomal enzymes), it does not follow that the system disclosed in the abovecited Patent Application be able to vehicle lysosomal enzymes in the apoplastic space of the seed storage tissue. For the abovementioned reasons, this localization could provide stability to expressed enzymes; in fact, an accurate localization of the lysosomal enzymes inside plant tissues is of fundamental importance for the stability of the produced enzyme.

Moreover, as it is known, unlike lactoferrin, enzymes are in most cases extremely unstable proteins requiring low-temperature storage, being easily inactivated by heat or denaturating agents.

In the light of these open questions, a person skilled in the art would hardly find and reckon in WO-A-00/04146 the technical support required to successfully express active lysosomal enzymes in storage organs of plant seeds.

Object of the present invention is to solve the problems left unsolved by the abovementioned state of the art. In particular, object of the present invention is to produce an expression system allowing the generation of transgenic plants able to express in seed a lysosomal enzyme being stable and in an enzymatically active form. A further object of the present invention is to produce such enzyme in an amount greater than that obtainable in leaf, i.e. greater than 1.5 mg per gram of tissue used.

### SUMMARY OF THE INVENTION

The invention is based on the unexpected discovery that lysosomal enzymes, of animal or human origin, can be advantageously expressed in seed storage organs in a form which is stable (stability exceeds 12 months in appropriately stored seeds), enzymatically active and in a high amount suitable for a medical use of said enzymes.

This amount is not lower than the 0.8%, preferably than the 1%. In an optimal form, the yield is of about the 1.5% of the total seed proteins, in order to use said seeds as storage and preservation means of such enzyme.

Moreover, regardless from the amount of expressed enzyme, the effective storage of the enzyme over lenghty periods (>12 months) enables to plan production at more favourable season periods, something not viable with in-leaf production, which requires immediate extraction and purification of the enzyme.

Within the present description, with the term “enzymatically active form” it is meant that the enzyme is functionally active and that its activity is equivalent to that of the native enzyme.

Hence, object of the present invention is a genetically transformed plant able to produce a lysosomal enzyme of animal or human origin, characterised in that:
said plant is transformed via the use of a recombinant expression vector comprising:
   a. a promoter of a plant gene specific for the expression in seed storage organs and stage-specific;
   b. a DNA sequence encoding the signal sequence of a plant protein able to dispatch said lysosomal enzyme to seed storage organs and to provide the post-translational modifications required for the expression of the enzyme in active form;
   c. a DNA sequence encoding said lysosomal enzyme deleted of the sequences encoding the signal sequence of the native enzyme and the sequence encoding the native polyadenylation signal;
   d. and a polyadenylation signal;
said enzyme is expressed in seed storage tissues in enzymatically active form and in an amount of at least the 0.8%, preferably of the 1%, of the total proteins of the seed.

Objects of the present invention are also the method for producing said plant by transforming plant cells with the abovedescribed vector and by regenerating plants from said transformed cells, the seeds produced by said plant, the method for producing said seeds, the method for purifying the enzyme from said seeds, the use of said seeds for the preparation of medicaments for enzyme replacement therapy and the use of said seeds as means for storing and preserving a lysosomal enzyme in enzymatically active form.

### DETAILED DESCRIPTION OF THE FIGURES

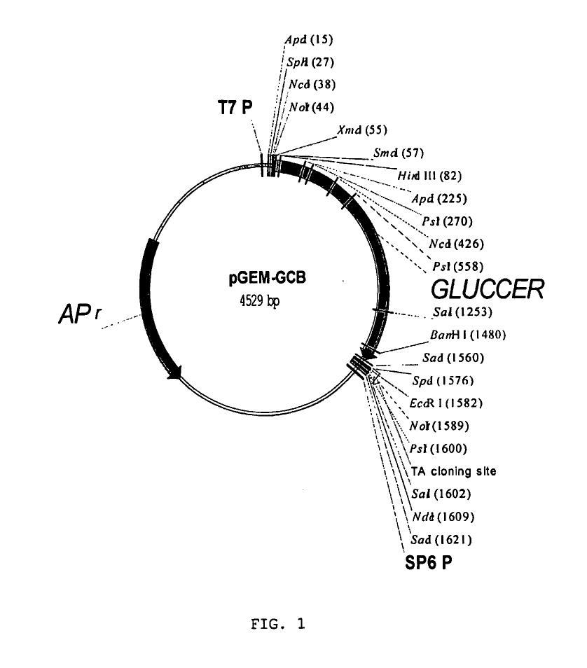

FIG. 1 shows the plasmid named pGEM-GCB obtained from the initial cloning of the GCB gene and used for the control of the complete sequence of the amplified gene. pGEM-GCB is obtained by cloning the fragment corresponding to the GCB gene having sequence SEQ ID NO 1 (obtained by cloning total RNA from human placenta by RT-PCR (reverse transcriptase PCR) with the primers having SEQ ID NO 3 and 4) amplified with the primers having SEQ ID NO 4 and 5 in pGEM®-T plasmid (Promega) in the EcoRV linearization site, and then delimiting the GCB gene between SacI and SmaI sites, in primers having SEQ ID NO 5 and 4, respectively. The primer having SEQ ID NO 5 allows the deletion of the DNA sequence portion encoding the human signal sequence from SEQ ID NO 1.

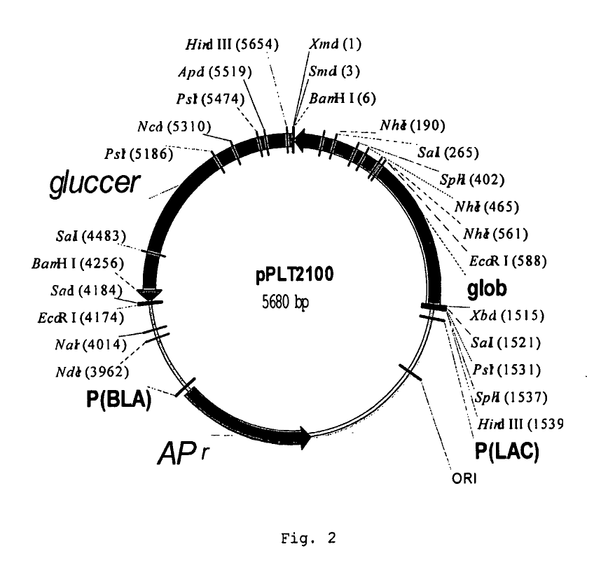

FIG. 2 shows the plasmid named pPLT2100, used to construct the PGLOB-GCB chimeric gene. pPLT2100 is obtained by inserting, between the SmaI and SacI sites of the pUC19 vector polylinker (EMBL Acc. N. X02514), the fragment corresponding to the GCB gene cloned in the plasmid of FIG. 1 cleaved with the same enzymes and the PGLOB promoter (SEQ ID NO 6) plus the sequence coding for the signal sequence of basic soy globulin (SEQ ID NO 7) cloned between the XbaI and BamHI sites.

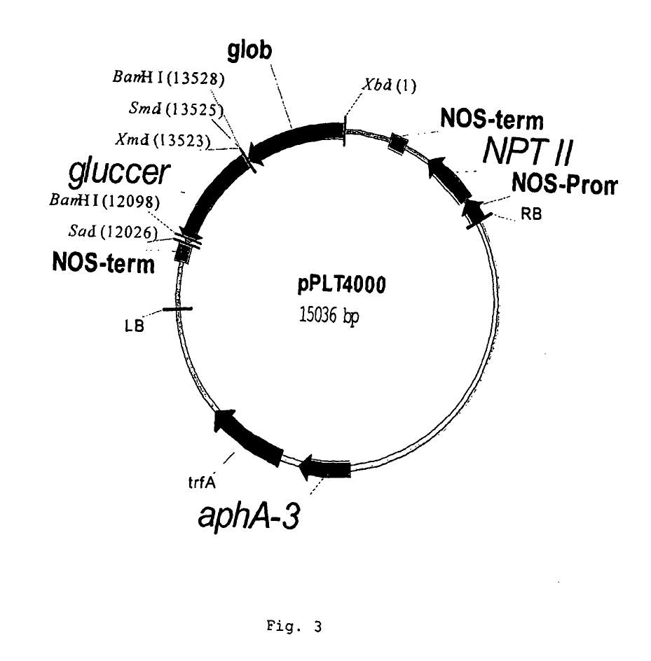

FIG. 3 shows the plasmid named pPLT4000 obtained by inserting between the XbaI-SacI sites of the pBI101 plasmid (Clontech) the PGLOB-GCB cassette cleaved with the same enzymes (XbaI-SacI) from the vector of FIG. 2.

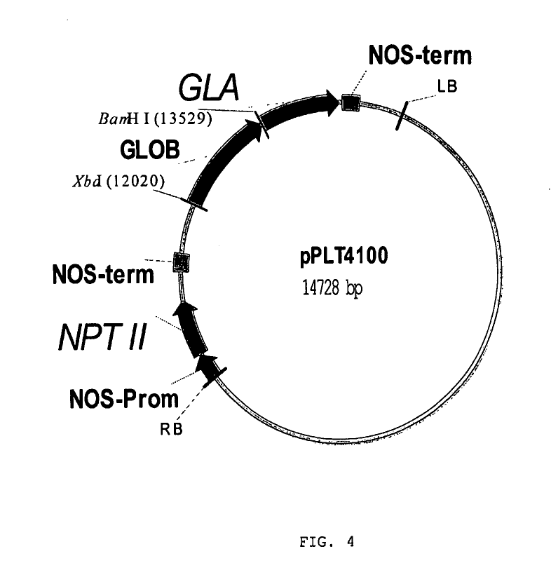

FIG. 4 shows the plasmid named pPLT4100 obtained by inserting between the BamHI-SacI (blunt) sites of the pBI101 plasmid (Clontech) the PGLOB-GLA cassette comprising the PGLOB promoter (SEQ ID NO 6), the signal sequence of the basic 7S soy globulin (SEQ ID NO 7) and the cDNA of the human □ galactosidase A gene (SEQ ID NO 8), indicated as GLA, deleted of the nucleotides coding for the signal peptide (i.e., deleted up to nucleotide 116). The PGLOB-GLA cassette was obtained by constructing plasmids analogous to those shown in FIGS. 1 and 2 in which the cDNA cloned is that of human α-galactosidase A (SEQ ID NO 8) deleted of the nucleotides encoding the signal peptide using the primers having sequences SEQ ID NO 10 and SEQ ID NO 11, which add a BamHI restriction site at 5′, and the ECORV (blunt) restriction site at 3′, of the amplified DNA, respectively.

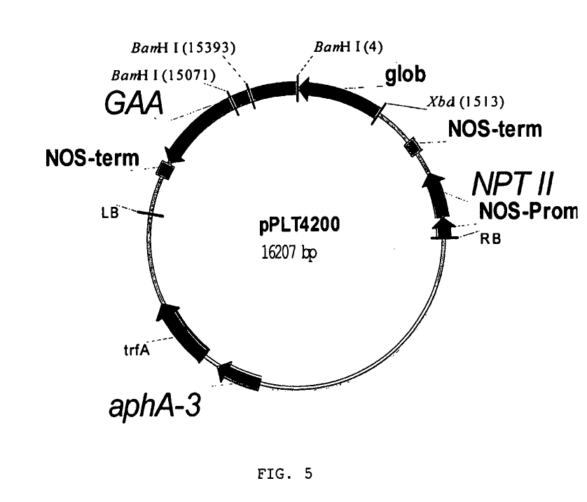

FIG. 5 shows the plasmid named pPLT4200 obtained by inserting between the SmaI-SacI sites (SacI is cleaved with the blunt EcoCR1 isoschizometer) of the pBI101 plasmid (Clontech) the PGLOB-GAA cassette comprising the PGLOB promoter (SEQ ID NO 6), the signal sequence of the basic 7S soy globulin (SEQ ID NO 7) and the cDNA of the human acid α-glucosidase gene (SEQ ID NO 12) indicated as GAA deleted of the nucleotides encoding the signal sequence (i.e. deleted up to nucleotide 426). The PGLOB-GAA cassette was obtained by constructing plasmids analogous to those shown in FIGS. 1 and 2 in which the cloned DNA is that of human acid α-glucosidase (SEQ ID NO 12), deleted of the nucleotides coding for the signal peptide using the primers having SEQ ID 14 and SEQ ID 15, which add the EcoRV (blunt) restriction site at 5′ and at 3′ of the amplified DNA.

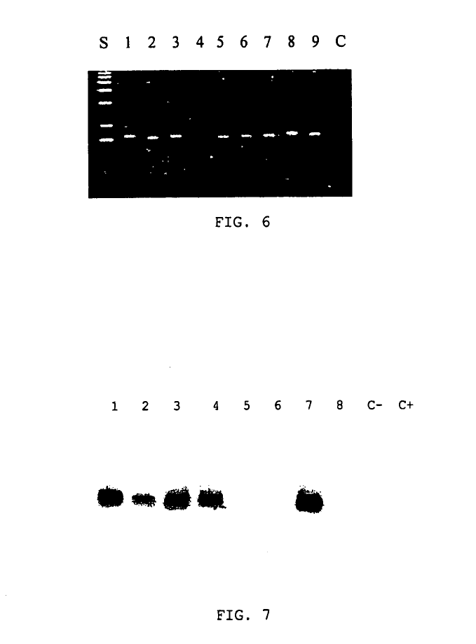

FIG. 6 reproduces the result of an agarose gel electrophoresis of the DNA from PCR amplification carried out with primers specific for the human glucocerebrosidase gene (SEQ ID NO 4 and 5), on DNA extracted from plants transformed with the construct of FIG. 3. The amplified fragment of about 1500 base pairs (bp) corresponds to the human glucocerebrosidase gene transformed in tobacco and present in the genome of nearly all the kanamycin-resistant tobacco lines obtained. The molecular weight marker ladder: 1 Kb ladder (Promega) (bands from bottom to top 1000 bp, 1500 bp, 2000 bp, 3000 bp, 4000 bp, 5000 bp, 6000 bp and 7000 bp) was loaded in the well of lane S. The product of DNA amplification of nine transformed tobacco plants was loaded in the wells of lanes 1 to 9. The product of the amplification carried out on the DNA of wild tobacco plants, not containing a homologous sequence amplifiable with the same primers, was loaded in the well of the C lane.

FIG. 7 reports the results of Northern analysis performed on RNA extracted from immature seeds (Days After Pollination, DAP, 20) of tobacco lines, transformed with the plasmid of FIG. 3 and tested positive at PCR control for gene presence detection, reported in FIG. 6. Total RNA extracted with the Trizol® method was loaded (10^{˜}μg/well) on agarose gel, subjected to electrophoresis, transferred on a nylon membrane and hybridised with a radioactive probe obtained by amplifying a human glucocerebrosidase gene fragment. Total RNA extracted from transgenic plants was loaded in lanes 1 to 8; C− is the negative control consisting of total RNA of a wild-type tobacco plant, C+ is the positive control consisting of total RNA (15□g) extracted from human placenta. The dimensions of the underlined band correspond to those expected for the messenger RNA (mRNA) of the GCB gene. The gene is not transcribed in plants 5, 6 and 8.

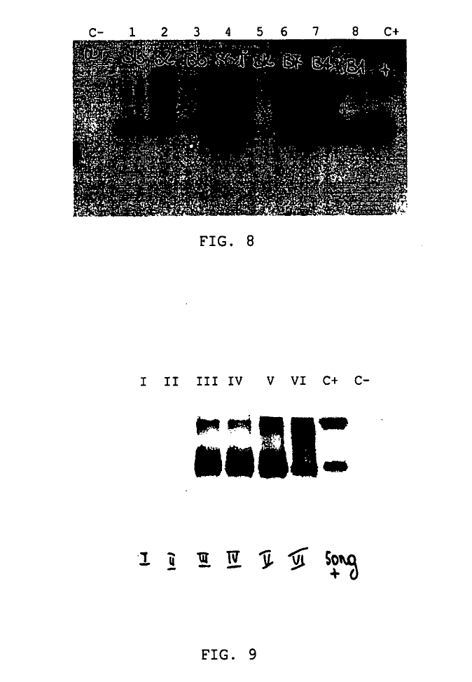

FIG. 8 reports a SDS-PAGE gel of proteins partially purified from seeds of transgenic tobacco plants transformed with the plasmid of FIG. 3 (lanes 1 to 8) reported in FIGS. 6 and 7, and their Western analysis. Western analysis was carried out on the total raw protein extract (1 mg) of mature seed, separated by SDS-PAGE electrophoresis. The analysis was carried out with chemiluminescence techniques, using a rabbit polyclonal antibody specific for human glucocerebrosidase enzyme as primary antibody, and a peroxidase-conjugated anti-rabbit IgG as secondary antibody. C− indicates the negative control, consisting of proteins extracted from wild-type tobacco seed; C+ indicates the positive control, consisting of commercial human glucocerebrosidase enzyme (50 ng).

FIG. 9 reports the expression profile of the PGLOB promoter in the tobacco seed of a transgenic line, regulating the expression of the GCB gene in the more productive lines. The different lanes contain total protein extracts obtained from the seed of a same line harvested at subsequent times after fertilization (DAP: day after pollination). Western Blot analysis, following the same procedure indicated for FIG. 8, ascertained the presence of the protein corresponding to the human glucocerebrosidase enzyme. Lane I: DAP 4; lane II: DAP 9; lane III: DAP 14; lane IV: DAP 18; lane V: DAP 22; lane VI: complete maturation; lane C+: positive control, commercial product 50 ng; lane C−: negative control, total protein extract from wild-type tobacco plants.

The results shown in this Figure highlight that in tobacco the soy PGLOB promoter has an activation profile which is optimal for the production of recombinant proteins, it being activated at about DAP 10 and expressed until maturation.

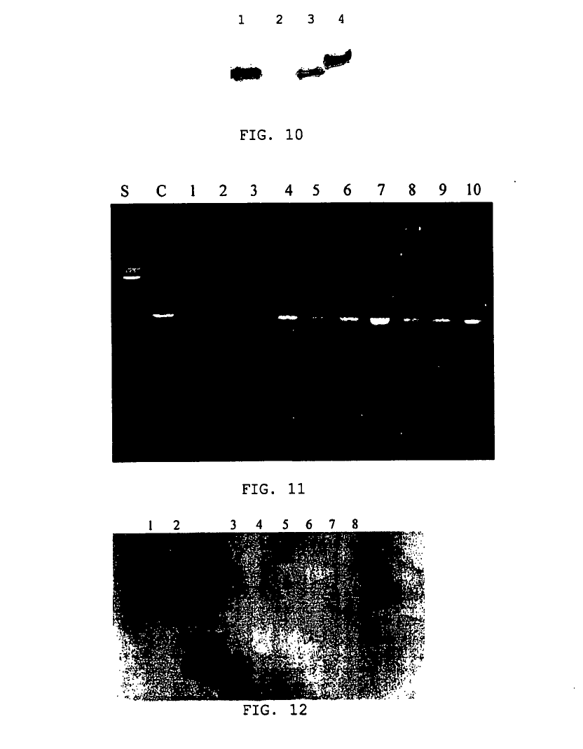

FIG. 10 reports the results of the deglycosilation enzyme treatment of the glucocerebrosidase protein purified from tobacco seed. Lane I: deglycosilated commercial human glucocerebrosidase enzyme, lane 2: control with proteins extracted from wild-type tobacco seed; lane 3: enzyme purified from seed and deglycosilated by deglycosilation enzyme treatment; lane 4: enzyme purified from seeds and not subjected to deglycosilation.

FIG. 11 reproduces the result of an agarose gel electrophoresis of the DNA resulting from PCR amplification, carried out with primers specific for the human α-galactosidase A gene (SEQ ID NO 10 and 11), on DNA extracted from plants transformed with the construct of FIG. 4. The amplified fragment of about 1200 bp corresponds to the human α-galactosidase A gene transformed in tobacco and present in the genome of nearly all the kanamycin-resistant tobacco lines obtained. The molecular weight marker ladder: 1 Kb ladder (Promega) (bands bottom to top 1000 bp, 1500 bp, 2000 bp, 3000 bp and 4000 bp) was loaded in the well of lane S. The product from DNA amplification of nine transformed tobacco plants was loaded in the wells of lanes lane 2-11. The product of the amplification carried out on the plasmid used for the transformation was loaded in the well of lane C.

FIG. 12. Western Blot related to the in-seed expression control of two tobacco lines transformed with GCB under control of the 35S promoter, and to the DNA sequence coding for the signal sequence of the human GCB gene. Western analysis was carried out with chemiluminescence techniques, using a rabbit polyclonal antibody specific for human glucocerebrosidase enzyme as primary antibody, and a peroxidase-conjugated anti-rabbit IgG as secondary antibody.

Lane 1: human glucocerebrosidase (50 ng), purified from placenta and deglycosilated; lane 2: human glucocerebrosidase (50 ng), non-deglycosilated and purified from placenta; lane 3: total proteins (1 mg) extracted from SR-S9 line seed; lane 4: total proteins (1 mg) extracted from SR-S10 line seed; lane 5: total proteins (1 mg) extracted from SR-S11 line seed; lane 6: total proteins (1 mg) extracted from SR-S12 line seed; lane 7: total proteins (1 mg) extracted from SR-S13 line seed; lane 8: total proteins (1 mg) extracted from SR-S14 line seed.

### DETAILED DESCRIPTION OF THE INVENTION

The lysosomal enzymes according to the present invention include but are not limited to: α-N-acetylgalactosaminidase, acid lipase, aryl sulfatase A, aspartylglycosaminidase, ceramidase, α-fucosidase, α-galactosidase A, α-galactosidase, galactosylceramidase, glucocerebrosidase, α-glucosidase, β-glucuronidase, heparin N-sulfatase, β-hexosaminidase, iduronate sulfatase, α-L-iduronidase, α-mannosidase, -mannosidase, sialidase and sphingomyelinase.

A person skilled in the art will know said enzymes, as well as their nucleotide and polypeptide sequences. The sequence encoding said enzymes might be isolated in accordance with any one known method. A preferred method for isolating said sequences is based on the RT-PCR technique. Suitable primers (including or not including the DNA sequence encoding the native signal sequence of the protein and the polyadenylation signal) may be designed from known sequences, and the cDNA of the desired gene may be cloned by RT-PCR from mRNA extracted from placental cells.

The cloned sequences including the DNA encoding the native signal sequence and/or the polyadenylation signal may subsequently be modified, by enzymatic treatments or by further PCR amplification using suitable primers, so as to delete said sequences.

In a preferred embodiment of the present invention, the lysosomal enzyme is human acid β-glucosidase (glucocerebrosidase or GCB). The cDNA of said enzyme (SEQ ID NO 1) may be cloned from mRNA extracted from placental cells using the primers having SEQ ID NO 3 and 4. Said cDNA, containing the sequence coding for the GCB native signal sequence, may further be PCR-amplified using the primers having SEQ ID NO 4 and 5, which delete said sequence, i.e. nucleotides 1-57 of SEQ ID NO 1, and adding restriction sites suitable for an easier insertion of the amplified product in the recombinant expression vector. The polyadenylation sequence is already absent from SEQ ID NO 1.

In another preferred embodiment of the present invention, the lysosomal enzyme is human α-galactosidase A (α-GalA or GLA). The cDNA of said enzyme (SEQ ID NO 8) already deleted of the portion encoding the signal peptide (i.e. to nucleotide 116), may be cloned from mRNA extracted from placental cells using the primers having SEQ ID NO 10 and 11. Said primers add restriction sites suitable for an easier insertion of the amplified product in the recombinant expression vector. The polyadenylation sequence is already absent from SEQ ID NO 8.

In yet another preferred embodiment of the present invention the lysosomal enzyme is human acid α-glucosidase (GAA). The cDNA of said enzyme (SEQ ID NO 12) already deleted of the portion encoding the signal peptide (i.e., to nucleotide 426), may be cloned from mRNA extracted from placental cells using the primers having SEQ ID NO 14 and 15. Said primers add restriction sites suitable for an easier insertion of the amplified product in the recombinant expression vector. The polyadenylation sequence is already absent in SEQ ID NO 12.

The recombinant expression vector for plant transformation of the present invention may be any one known vector suitable for the transformation of plant cells and for the expression of protein products in them. Said vector may be cleaved at the most appropriate restriction sites, and in it, an expression cassette according to the present invention may be inserted.

Suitable plant transformation vectors according to the present invention include, but are not limited to, Agrobacterium Ti plasmids and derivatives thereof, including both integrative and binary vectors, plasmids pBIB-KAN, pGA471, pEND4K, pGV3850, pMON505 and pBI101. Included among the vectors of the present invention are also DNA or RNA plant viruses like, e.g., cauliflower mosaic virus, tobacco mosaic virus and their engineered derivatives genetically suitable for the expression of lysosomal enzymes. Moreover, transposable elements may be used in conjunction with any vector to transfer the expression cassette of the present invention into the plant cell.

Preferred vector for the purposes of the present invention is the expression plasmid pBI101 in which an expression cassette is inserted.

The expression cassette according to the present invention comprises a promoter of a plant gene, stage-specific and specific for the expression in seed storage organs. As stage-specific, it is meant a promoter inducing the expression of the gene it controls at a specific seed development stage.

A promoter suitable for practicing of the present invention is the promoter of the basic 7S soy globulin (SEQ ID NO 6). The expression cassette according to the present invention further comprises a DNA sequence coding for the signal sequence of a plant protein able to dispatch said lysosomal enzyme to the seed storage organs and to ensure the post-translational modifications required for the expression of the enzyme in enzymatically active form.

A signal sequence suitable for practicing the present invention is the signal sequence of basic 7S soy globulin (SEQ ID NO 7).

The expression cassette according to the present invention also contains a DNA sequence encoding a lysosomal enzyme of the ones listed above, deleted of the sequences encoding the native signal sequence and the polyadenylation signal.

Moreover, the expression cassette of the present invention contains a polyadenylation signal (or that already present in the expression vector can be used).

The elements constituting the expression cassette of the present invention should be functionally linked, in the aboveindicated order in the 5′->3′ direction.

Optionally, the nucleotide sequence encoding the enzyme may be preceded by a short sequence apt to ease purification of the same protein on affinity columns, like e.g., his 6× tag, FLAG® (Sigma) or GST (Amersham).

In a preferred embodiment of the present invention, the vector derives from pBI101 plasmid (Clontech) in which the abovedescribed expression cassette is inserted. Said cassette comprises, besides the DNA encoding the desired lysosomal enzyme, the promoter of the basic 7S soy globulin gene (PGLOB) (SEQ ID NO 6) and the DNA coding for the signal sequence of the gene of the basic 7S soy globulin (SEQ ID NO 7). Said expression cassette, inserted in pBI101 vector (Clontech) upstream of the polyadenylation signal already present in the vector, contains the PGLOB promoter and the DNA sequence encoding the signal sequence of the basic 7S globulin fused to the cDNA of the human lysosomal enzyme (e.g., GCB SEQ ID NO 1, α-GalA SEQ ID NO 8 and GAA SEQ ID NO 12) deleted of nucleotides coding for the signal sequence of the native enzyme and of any nucleotide not coding the mature enzyme; i.e., in the case of SEQ ID NO 1 nucleotides 1-57, in the case of SEQ ID NO 8 nucleotides 1-116 and, in the case of SEQ ID NO 12, nucleotides 1-426.

Plants suitable for practicing the present invention are those with seeds exhibiting a high protein content, among which Leguminosae, cereals and tobacco are particularly suitable.

In fact, as indicated above, protein content in seeds is of about the 25-35% in Leguminosae, of about the 10-15% in cereals and of about the 20% in tobacco.

In the preferred embodiment of the present invention tobacco plant was used.

The transformation of the plant cells from which the plant according to the present invention is regenerated may be carried out by any one technique known to a person skilled in the art, like Agrobacterium-mediated transformation of leaf discs or of other plant tissues, microinjection of DNA directly into plant cells, electroporation of DNA into plant cell protoplasts, liposome or spheroplast fusion, microprojectile bombardment and the transfection of plant cells or tissues with appropriately engineered plant viruses.

The invention can be practiced by transforming or transfecting plant cells with recombinant expression vectors containing the expression cassette according to the present invention and selecting the transformants or transfectants expressing the enzyme. The transformants may be selected by selection markers commonly known in the state of the art. Plant transformants are selected and induced to regenerate fertile whole plants able to form seeds expressing the lysosomal gene in enzymatically active form following agritechnical methods known in the specific field. In the preferred embodiment of the present invention, the transformation of the plant cells from which the plant is regenerated is carried out with Agrobacterium cells made competent by electroporation. The strain with the vector is used to transform leaf discs (LD). Formation first of shoots and then of roots was induced from calluses formed on LD in the presence of selective antibiotic. The plant genetically transformed according to the present invention is a stable transgenic plant whose genetic information, inserted subsequently to the tranforming, is present and expressed (without gene silencing phenomena) in the subsequent generations.

The method for extracting the enzyme produced in seed according to the present invention may be any one standard method for extracting protein from plant tissues known to a person skilled in the art. Said method provides seed grinding in liquid nitrogen in a suitable buffer, centrifuging, supernatant recovering, filtering and further enzyme purification by normal or affinity chromatography.

In a preferred embodiment, the extraction buffer used consists of: sucrose, ascorbic acid, Cys-HCl, Tris-HCl, pH6 EDTA; centrifuging takes place at 2-10° C., preferably at 4° C., at 14,000 rpm for 5-60 min, preferably for 30 min, filter porosity is of about 0.2 μm and the HPLC chromatography column is any column at a weak cationic exchange suitable for the purpose. Preferably, lane Resource Q column (Pharmacia), at a weak cationic exchange with elution in phosphate buffer pH 6 and NaCl gradient 20-100%, is used. For the affinity column, the specific antibodies are bound to affigel-type resins (Biorad) and the elution is carried out with ethylene glycol.

The enzyme extracted from the seed may be used for the preparation of medicaments suitable for enzyme replacement therapy. Therefore, preferably said enzyme will exhibit a high concentration in seed, i.e. between the 0.8 and the 1.5%, preferably between the 0.8 and the 1%.

Optionally, since the in-plant produced enzyme could contain N-linked glycans different from those present in a human product, said enzyme could be modified post-extraction in order to avoid any immunogenic reactions in a patient requiring a regular infusion of the glycoprotein. In vitro modifications could remove the xylose residue normally bound to the mannose of plant-produced glycans, according to techniques already used on proteins produced with a CHO cell-based system.

The following examples are meant to provide a more detailed description of the invention without however limiting to them the object that is being claimed.

### EXAMPLES

### Example 1

Construction of the Vector for the Expression of Human Glucocerebrosidase in Plant Seeds.

The GCB gene coding for human glucocerebrosidase was cloned, by RT-PCR technique, from mRNA purified from placenta with primers having SEQ ID NO 3 and 4. The gene was isolated, amplifying the DNA having SEQ ID NO 1 with the primers having SEQ ID NO 4 and 5, in its structural portion deleted of the signal peptide (i.e., deleted of the signal sequence of the human gene, namely the nucleotides 1-57 of SEQ ID NO 1) and of the poly-A site (which is not amplified by said primers) and cloned in pGEM®-T (Promega) to form the plasmid named pGEM-GCB (FIG. 1). The primers designed for amplification add the SmaI restriction site at 5′ and the SacI restriction site at 3′. The sequence of the natural gene obtained, which at sequence control tested identical to the published one (Sorge et al., 1985, Proc. Natl. Acad. Sci. 82: 7289-7293), was cloned in the vector named pPLT2100, in the SmaI and SacI sites, under control of the PGLOB promoter (FIG. 2) to form the plasmid named pPLT4000 (FIG. 3). After accurate control by restriction, the resulting pPLT4000 plasmid was used for genetic transformation of plants.

### Example 2

Genetic Transformation of Plants with pPLT4000 Plasmid and General Results

pPLT4000 plasmid was transferred in A. tumefaciens strain EHA105 cells, made competent, by electroporation. The strain with the plasmid was used to transform about 300 leaf discs of tobacco cultivar (cv) Xanthi. From calluses generated onto leaf discs in the presence of kanamycin first shoot and then root initiation was induced. Rooted plantlets were potted, and at least 150 kanamycin-resistant plants were analysed.

Plants T₀ were tested by PCR (FIG. 6) and plants T₁ by Northern (FIG. 7) and Western (FIG. 8) analyses.

All plants with GCB gene under control of PGLOB promoter led to accumulation of a protein, recognised by GCB-specific antibodies, having a molecular weight equal to 58 kDa corresponding to the glycosilated human protein (FIG. 8). The presence of the recombinant protein exclusively in the seed and not in the leaves was assayed in all the examined transgenic plants.

Recombinant GCB protein isolated from seed and purified with HPLC techniques showed to be identical to the natural protein with concern to the molecular weight. Treatment with deglycosilation enzymes confirms the presence of posttranslational modifications in all alike, at present at least in quantitative terms, those present in native glucocerebrosidase (FIG. 10).

### Example 3

Agrobacterium Tumefaciens-Mediated Tobacco Transformation

Day 1: a small amount of Agrobacterium tumefaciens of strain EHA 105, taken from a Petri plate culture with a sterile loop so as not to exceed in the amount, thereby avoiding subsequent problems in controlling bacterial proliferation on plated leaf discs, was inoculated in 2 ml of sterile LB. Then, from a healthy tobacco plant cv Xanthi a leaf showing no alteration whatsoever, conversely exhibiting optimal turgor conditions, was taken. The leaf was briefly rinsed with bidistilled water to remove surface impurities, immersed for 8 min in a 20% sodium hypochlorite and 0.1% SDS solution and left to dry under a vertical flow hood. From then on, all steps were carried out under hood. In particular, the leaf was immersed in 95% ethanol and shaken in order to drench its two pages (letting the petiole emerge) for 30-40 sec. The leaf was then allowed to dry out completely.

Discs were obtained from the entire leaf surface with an ethanol-sterilised punch, let fall on plates with antibiotic-free MS10; in particular, the ratio of 30 discs per plate was not exceeded. Next, 2 ml LB+(just inoculated) Agrobacterium were poured on plate, and the bacterial suspension was evenly spread over the entire plate with a gentle rotary motion, in order to obtain an homogeneous bacterial distribution among the discs. LB in excess was carefully aspirated with a pipette. At all times in the course of those steps a parallel negative control was performed by means of a plate to which nothing, or only LB was added.

Then plates were incubated at 28° C. for 24-48 hours under constant lighting conditions, and bacterial growth was indicated by the appearance of a thin opaque layer spreading over the entire plate.

Day 2

Leaf discs (LD) were carefully transferred on a plate with MS10+500 mg/l cephotaxime, and incubated at 28° C. for 6 days under constant lighting conditions. This step determines Agrobacterium inactivation.

Day 8

LD were then carefully transferred on a plate with MS10+500 mg/l cephotaxime and 200 mg/l kanamycin, and incubated at 28° C. for 14 days, under constant lighting conditions. This step determined a selection of the transformed plants: in fact, kanamycin resistance gene was carried by the plasmid inserted into Agrobacterium.

Day 22

LD, that in the meantime had grown developing a callus, were carefully transferred on a plate with MS10+500 mg/l cephotaxime 500 mg/l, 200 mg/l kanamycin and 500 mg/l carbenicillin, and incubated for 6 days. This step determines elimination of the Agrobacteria possibly survived to previous antibiotic treatments (a very frequent occurrence).

Day 28

LD were transferred again on MS10+500 mg/l cephotaxime and 200 mg/l kanamycin, and incubated until shoot appearance. Then, shoots exhibiting at least two leaves were separated from the callous mass and transferred in the radication medium: MSO+500 mg/l cephotaxime and 200 mg/l kanamycin.

At root appearance, the seedlings were extracted from the plate, freed from agar residues, gently rinsed with running water and planted out in loam and sand (2:1) inside small plastic pots. Soil was previously saturated with water and then pots were covered with transparent plastic lids to preserve high humidity conditions, and placed in a growth chamber at 25° C., with a daily 16-hour lighting period.

The presence of the construct containing the DNA coding for the GCB was assayed by PCR in plants T₀ (FIG. 6) and, subsequently, in plants T₁-T₅. The presence of the transcript was assayed by Northern blotting in plants T₁ (FIG. 7) as well as the presence of the protein by Western blotting (FIG. 8) and, subsequently, in plants T₂ to T₆. The presence of the construct in generations 0-5 and of the transcript as well as of the protein in generations 1-6 demonstrates the stability of the transformation effected, and the absence of gene silencing phenomena in all examined generations subsequent to the first one.

### Example 4

Purification of Glucocerebrosidase Protein from Different Tissues of the Plant and Assessment of Molecular Weight.

Extraction of all the tobacco seed proteins was performed grinding the seeds in liquid nitrogen in the presence of an extraction buffer (0.5 M sucrose, 0.1% ascorbic acid, 0.1% Cys-HCl, 0.01 M Tris-HCl, 0.05M EDTA pH 6).

Then the resulting solution was centrifuged at 14.000 rpm for 30 min at 4° C. and the supernatant with the soluble proteins was recovered.

The solution was filtered with filters of 0.2 μm porosity, and the glucocerebrosidase partially purified by removing proteins of a <30 KDa molecular weight by centrifugation in Centricon 30 column (Amicon).

The glucocerebrosidase was further purified by HPLC chromatography on Resource Q column (Pharmacia) at a weak cationic exchange, with elution in phosphate buffer pH 6 and NaCl gradient 20-100%. The peak corresponding to glucocerebrosidase eluted at 0.6 M NaCl.

The elution fractions were reunited and filtered in Centricon 30 to remove salt.

For the glucocerebrosidase extraction from tobacco seeds, up to the centrifugation step the Inventors proceeded as in the case of extraction from seed, then the supernatant was additioned with 60% (NH₄)₂SO₄ and left shaking in ice for 60 min.

Then the solution was centrifuged at 14,000 rpm for 15 min at 4° C., the pellet recovered and then resuspended in phosphate buffer pH 6.8.

For the assessment of molecular weight in SDS-PAGE, the staining agent (SDS loading buffer) was additioned to the glucocerebrosidase sample (20 μl) and the samples were loaded onto 10% polyacrylamide minigels. Running conditions were: initially 10 mA, and 20 mA for the entire run, in Tris-glycine 1× buffer. Then the gel was stained by Silver staining technique and the molecular weight assessed referring to molecular weight standards.

### Example 5

Western Analysis of the Glucocerebrosidase Protein Produced in Plant and Deglycosilation Thereof.

Glucocerebrosidase purified from seed according to example 5, after electrophoretic separation on acrylamide gel, was transferred by electroblotting (buffer 25 mM Tris, 192 mM glycine, 20% methanol, 45 V at 4° C.) onto a nitrocellulose membrane (BA85 Schleicher and Schull).

The membrane with the immobilised protein was shaken for 60 min in TBS-T 5% Skim milk solution and then, after at least three rinsings with TBS-T, the membrane was incubated for 60′ at room temperature, shaken with TBS-T 5% Skim milk and primary antibody (rabbit polyclonal antibody specific for human GCB) in a 1:2500 ratio.

After reaction with the primary antibody, the membrane was rinsed at least 3 times with TBS-T and then incubated, shaken for 60′ with the secondary antibody (Peroxidase-conjugated anti-rabbit IgG), always in TBS-T 5% Skim milk solution, in a 1:12,000 ratio.

After reaction with the secondary antibody, the membrane was rinsed at least 3 times with TBS-T and placed in contact with Amersham's chemiluminescence kit ECL™ solutions.

Then the membrane was exposed in contact with a photoplate (Hyperfilm™ MP, Amersham) in darkroom for variable lengths of time (FIG. 8).

Deglycosilation with N-glycosidase A and F enzymes (Calbiochem, Boehringer Mannheim) was carried out using 10 μl b/v of glycopeptide (10 μg) denatured in 0.1% SDS brought to boiling point for 2 min.

90 μl of buffer (20 mM phosphate buffer pH 7.2, 50 mM EDTA pH 8, 10 mM sodium azide, 0.5% NP40, 1% β-mercaptoethanol) were additioned to this solution, which was brought to boiling point again for 2 min, then cooled at 37° C. To the resulting 100 μl, 1 U of N glycosidase F and 1 U of N glycosilase A were additioned, and let incubate at 37° C. for 18 hours. Then the reaction product was analysed on SDS-PAGE gel and the glucocerebrosidase protein detected by Western technique (FIG. 10).

### Example 6

Assessment of Enzymatic Activity of the Protein Produced and Accumulated in Seed.

The enzymatic assay was carried out in 200 μl of an 100 mM buffer K phosphate, pH 5, 0.15% triton x-100, 0.125% Na taurocolate, 4-MUG (4 methyl-umbelliferyl glucopyranoside) solution, at 37° C. for 60′.

The reaction was terminated by addition of 1 ml of a 0.1M glycine solution pH 10 and the reading was carried out at the spectrophotometer with 340 and 448 nm wavelengths. The calibration curve of the instrument was effected using a commercial enzyme product. For each sample, the assay was repeated in triplicate using 250 ng of partially purified protein. The results are reported in Table 1.

TABLE 1
β-glucocerebrosidase enzymatic activity
measured in total extracts of proteins from tobacco seed.

| Tobacco line | Fluorescence |
|---|---|
| WT | 1.4 ± 0.6 |
| SG1 | 26.1 ± 1.5 |
| SG2 | 34.7 ± 1.8 |
| SG10 | 37.0 ± 1.5 |
| SG3 | 21.4 ± 0.9 |
| SG4 | 22.5 ± 1.1 |
| SG17 | 43.9 ± 1.7 |
| SG20 | 18.8 ± 1.2 |
| SG22 | 21.8 ± 1.4 |
| SG31 | 28.9 ± 1.6 |
| SGB | 19.2 ± 1.0 |

### Example 7

Quantitative Determination of the Protein

The assessment of human GCB accumulation capability of the various transgenic lines was carried out comparing the amount of GCB protein in seed to known amounts of commercial GCB. Protein amount was determined after electrophoretic separation on SDS-PAGE gel and detection with a polyclonal primary antibody, produced in rabbit and specific for human GCB and with a peroxidase-conjugated anti-rabbit IgG secondary antibody. The specific band was detected with a scanner, and the protein amount was determined comparing the intensity of the band to that of bands containing a known amount of protein purified and present in the same gel at different dilutions. Thus, tobacco lines were identified whose protein extract produces 8-10 μg GCB per mg of seed-extracted total proteins, i.e. corresponding to an amount ranging from the 0.8% to the 1% of the total proteins of the seed.

### Example 8

Construction of the Vector for the Expression of Human α-Galactosidase A in Plant Seeds.

The GLA gene coding for human α-galactosidase A was cloned, by RT-PCR technique, from mRNA purified from placenta, with primers having SEQ ID NO 10 and 11. The gene was isolated, in its structural portion also deleted of the signal peptide (i.e. deleted of nucleotides 1-116 of SEQ ID NO 8) and of the poly-A site (not amplified by said primers) by amplifying the DNA having SEQ ID NO 8 starting from nucleotide 117 with the same primers, and cloned in pGEM®-T (Promega) to form the plasmid named pGEM-GLA. The primers designed for the amplification add the BamHI restriction site at 5′ and the EcoRV restriction site at 3′. The natural gene obtained, which at sequence control tested identical to the published one (Tsuji S. et al., 1987, Eur. J. Biochem., 165(2), 275-280) in the portion encoding the mature peptide, was cloned in a vector analogous to the one named pPLT2100 of example 1 under control of the PGLOB promoter to form the plasmid named pPLT4100 (FIG. 4). After accurate control by restriction, the resulting pPLT4100 plasmid was used for genetic transformation of plants.

### Example 9

Genetic Transformation of Plants with pPLT4100 Plasmid

Tobacco plant transformation was carried out using the vector named pPLT4100 according to the same methodologies used in examples 2 and 3.

The presence of the construct containing the DNA coding for GLA was assayed by PCR in plants T₀ (FIG. 11).

### Example 10

Construction of the Vector for the Expression of Human Acid α-Glucosidase in Plant Seeds.

The GAA gene coding for human acid □-glucosidase was cloned deleted of the region encoding the signal peptide, by RT-PCR technique, from mRNA purified from placenta, with the primers having sequences SEQ ID NO 14 and 15. The gene, in its structural portion deleted of the signal peptide as well (i.e. deleted of nucleotides 1-426 of SEQ ID NO 12) and of the poly-A site (not amplified by said primers) was isolated, amplifying the DNA having SEQ ID NO 12 starting from nucleotide 427 with the same primers, and cloned in pGEM®-T (Promega) to form the plasmid named pGEM-GLA. The primers designed for the amplification add the restriction site EcoRV to 5′ and to 3′. The natural gene obtained, which at sequence control tested identical to the published one (Hoefsloot L. H. et al., 1988, EMBO JOURNAL 7(6), 1697-1704) in the portion encoding the mature peptide, was cloned in a vector analogous to that named pPLT2100 of example 1 under control of the PGLOB promoter to form the plasmid named pPLT4200 (FIG. 5). After accurate control by restriction, the resulting pPLT4200 plasmid was used for genetic transformation of plants.

### Comparative Example

Absence of GCB Expression in Seed Using the ³⁵S Promoter of the Cauliflower Mosaic Virus

In order to assess operation of the 35S promoter of the Cauliflower mosaic virus indicated in U.S. Pat. No. 5,929,304 as promoter suitable for expressing lysosomal enzymes, tobacco plants were transformed using a recombinant expression vector comprising the sequence SEQ ID NO 1 under control of the promoter 35S. The proteins were extracted according to standard methods and the protein product was analysed by Western technique under chemiluminescence using a human GCB-specific rabbit polyclonal antibody as primary antibody, and a peroxidase-conjugated anti-rabbit IgG as secondary antibody. The results, reported in FIG. 11, demonstrate that using the 35S promoter for expressing human glucocerebrosidase no accumulation of stable protein in the seed is obtained.

1-22. (canceled)

23. A genetically transformed plant able to produce a lysosomal enzyme of animal or human origin, said plant transformed via the use of an expression vector comprising:
a. a promoter of a plant gene specific for the expression in seed storage organs and stage-specific;
b. a DNA sequence encoding the signal sequence of a plant protein able to dispatch said lysosomal enzyme to seed storage organs and to provide the post-translational modifications required for the expression of the lysosomal enzyme in active form; and
c. a DNA sequence encoding said lysosomal enzyme deleted of the native signal sequence;
wherein said lysosomal enzyme is expressed in seed storage tissues in enzymatically active form and in an amount of at least the 0.8% of the total proteins of the seed.

24. The plant according to claim 23, wherein the expression vector is a plasmid.

25. The plant according to claim 23, wherein the promoter is a 7S soy globulin gene promoter or a functional equivalent.

26. The plant according to claim 23, wherein the signal sequence is a 7S soy globulin signal sequence, or a functional equivalent, and is fused to the sequence encoding the structural portion of the mature lysosomal enzyme deleted of the native signal sequence.

27. The plant according to claim 25, wherein the signal sequence is a 7S soy globulin signal sequence, or a functional equivalent, and is fused to the sequence encoding the structural portion of the mature lysosomal enzyme deleted of the native signal sequence.

28. The plant according to claim 23, wherein the lysosomal enzyme expressed in enzymatically active form in seed storage tissues is selected from the group consisting of α-N-acetylgalactosaminidase, acid lipase, aryl sulfatase A, aspartylglycosaminidase, ceramidase, α-fucosidase, α-galactosidase A, β-galactosidase, galactosylceramidase, glucocerebrosidase, α-glucosidase, β-glucuronidase, heparin N-sulfatase, β-hexosaminidase, iduronate sulfatase, α-L-iduronidase, α-mannosidase, β-mannosidase, sialidase, and sphingomyelinase.

29. The plant according to claim 27, wherein the lysosomal enzyme expressed in enzymatically active form in seed storage tissues is selected from the group consisting of α-N-acetylgalactosaminidase, acid lipase, aryl sulfatase A, aspartylglycosaminidase, ceramidase, α-fucosidase, α-galactosidase A, β-galactosidase, galactosylceramidase, glucocerebrosidase, α-glucosidase, α-glucuronidase, heparin N-sulfatase, α-hexosaminidase, iduronate sulfatase, α-L-iduronidase, α-mannosidase, β-mannosidase, sialidase, and sphingomyelinase.

30. The plant according to claim 23, wherein said plant is a Leguminosa, a cereal, or tobacco.

31. The plant according to claim 29, wherein said plant is a Leguminosa, a cereal, or tobacco.

32. A method for producing a genetically transformed plant able to produce a lysosomal enzyme, said enzyme being expressed in seed storage tissues of said plant in an enzymatically active form and in an amount of at least the 0.8% of the total proteins of the seed, comprising the following steps:
constructing an expression vector comprising:
a. a promoter of a plant gene specific for the expression in seed storage organs and stage-specific;
b. a DNA sequence encoding the signal sequence of a plant protein able to dispatch said lysosomal enzyme to seed storage organs and to provide the post-translational modifications required for expression of the enzyme in active form; and
c. a DNA sequence encoding said lysosomal enzyme deleted of the native signal sequence;
transforming plant cells with said vector; and
using said cells to regenerate said transformed plant.

33. The method according to claim 32, wherein said plant is a Leguminosa, a cereal, or tobacco.

34. A seed of genetically modified plant able to express a lysosomal enzyme, wherein:
said seed contains an expression vector comprising:
a. a promoter of a plant gene specific for the expression in seed storage organs and stage-specific;
b. a DNA sequence encoding the signal sequence of a plant protein able to dispatch said lysosomal enzyme to seed storage organs and to provide the post-translational modifications required for the expression of the enzyme in active form; and
c. a DNA sequence encoding said lysosomal enzyme deleted of the native signal sequence;
said enzyme is contained in seed storage tissues in enzymatically active form and in the amount of at least the 0.8% of the seed total proteins.

35. The seed according to claim 34, wherein the expression vector is a plasmid.

36. The seed according to claim 34, wherein the promoter is a 7S soy globulin gene promoter or a functional equivalent.

37. The seed according to claim 34, wherein the signal sequence is a 7S soy globulin signal sequence, or a functional equivalent, and is fused to the sequence encoding the structural portion of the mature lysosomal enzyme deleted of the native signal sequence.

38. The seed according to claim 36, wherein the signal sequence is a 7S soy globulin signal sequence, or a functional equivalent, and is fused to the sequence encoding the structural portion of the mature lysosomal enzyme deleted of the native signal sequence

39. The seed according to claim 34, wherein the lysosomal enzyme expressed in enzymatically active form in seed storage tissues is selected from the group consisting of:
α-N-acetylgalactosaminidase, acid lipase, aryl sulfatase A, aspartylglycosaminidase, ceramidase, α-fucosidase, α-galactosidase A, β-galactosidase, galactosylceramidase, glucocerebrosidase, α-glucosidase, β-glucuronidase, heparin N-sulfatase, β-hexosaminidase, iduronate sulfatase, α-L-iduronidase, α-mannosidase, β-mannosidase, sialidase, and sphingomyelinase.

40. The seed according to claim 38, wherein the lysosomal enzyme expressed in enzymatically active form in seed storage tissues is selected from the group consisting of α-N-acetylgalactosaminidase, acid lipase, aryl sulfatase A, aspartylglycosaminidase, ceramidase, α-fucosidase, α-galactosidase A, β-galactosidase, galactosylceramidase, glucocerebrosidase, α-glucosidase, β-glucuronidase, heparin N-sulfatase, β-hexosaminidase, iduronate sulfatase, α-L-iduronidase, α-mannosidase, β-mannosidase, sialidase, and sphingomyelinase.

41. The seed according to claim 34, wherein said seed is of a Leguminosa, a cereal or tobacco.

42. The seed according to claim 40, wherein said seed is of a Leguminosa, a cereal or tobacco.

43. A method for producing a seed of genetically modified plant able to express a lysosomal enzyme, said enzyme being contained in said seed storage tissues in an enzymatically active form and in the amount of at least the 0.8% of said seed total proteins, comprising the following steps:
constructing an expression vector comprising:
a. a promoter of a plant gene specific for the expression in seed storage organs and stage-specific;
b. a DNA sequence encoding the signal sequence of a plant protein able to dispatch said lysosomal enzyme to seed storage organs and to provide the post-translational modifications required for expression of the enzyme in active form; and
c. a DNA sequence encoding said lysosomal enzyme deleted of the native signal sequence; and
transforming plant cells with said vector; and using said cells to regenerate transformed plants able to produce said seeds.

44. The method according to claim 43, wherein said seed is of a Leguminosa, a cereal or tobacco.

45. A method for extracting and purifying the lysosomal enzyme in active form contained in the seed of a genetically modified plant able to express a lysosomal enzyme, said enzyme being contained in said seed storage tissues in an enzymatically active form and in the amount of at least the 0.8% of said seed total proteins, comprising the following steps:
a. grinding said seed in liquid nitrogen in the presence of an extraction buffer;
b. centrifuging the resulting solution;
c. recovering and filtering the supernatant with filters having a porosity suitable to the enzyme dimensions; and
d. further purifying the partially purified enzyme by HPLC chromatography.

46. A method of use of a seed of a genetically modified plant able to express a lysosomal enzyme, said enzyme being contained in said seed storage tissues in an enzymatically active form and in the amount of at least the 0.8% of said seed total proteins, comprising preparing a medicament for enzyme replacement therapy that comprises the lysosomal enzyme extracted from said seed.

47. The method use of the seed according to claim 46 for the preparation of a medicament for an enzyme replacement therapy in Gaucher disease.

48. The method of use of the seed according to claim 46 for the preparation of a medicament for an enzyme replacement therapy in Anderson-Fabry disease.

49. The method of use of the seed according to claim 46 for the preparation of a medicament for an enzyme replacement therapy in Pompe disease.

50. A method of use of a seed of a genetically modified plant able to express a lysosomal enzyme, comprising the steps of:
a. transforming a plant with an expression vector of claim 1 to yield the genetically modified plant;
b. growing the genetically modified plant;
c. harvesting the seed of the genetically modified plant;
d. purifying the lysosomal enzyme from the seed; and
e. preparing a medicament for enzyme replacement therapy that comprises the purified lysosomal enzyme from step “e”.

51. A method of use of a seed of a genetically modified plant able to express a lysosomal enzyme, said enzyme being contained in said seed storage tissues in an enzymatically active form and in the amount of at least the 0.8% of said seed total proteins, comprising and preserving the lysosomal enzyme in enzymatically active form by storing said seed.
